(19) United States
(12) Patent Application Publication
(10) Pub. No.: US 2025/0009747 A1
(43) Pub. Date: Jan. 9, 2025

(54) ENDOTHELIN RECEPTOR ANTAGONISTS FOR USE IN TREATING OR PREVENTING MUSCLE FIBROSIS

(71) Applicants: ASSOCIATION INSTITUT DE MYOLOGIE, PARIS (FR); SORBONNE UNIVERSITE, PARIS (FR); INSERM (INSTITUT NATIONAL DE LA SANTÉ ET DE LA RECHERCHE MÉDICALE), PARIS (FR)

(72) Inventors: MONA BENSALAH, PARIS (FR); ANNE BIGOT, FONTENAY SOUS BOIS (FR); LAURA MURAINE, GENTILLY (FR); ELISA NEGRONI, PARIS (FR); CAPUCINE TROLLET, PARIS (FR)

(21) Appl. No.: 18/704,956

(22) PCT Filed: Oct. 27, 2022
(86) PCT No.: PCT/EP2022/080136

§ 371 Date: Apr. 26, 2024

(30) Foreign Application Priority Data
Oct. 28, 2021 (EP) 21306516.2

(51) Int. Cl. A61K 31/506; A61K 31/454; A61K 38/08; A61P 21/00; G01N 33/68; G01N 33/74

(52) U.S. Cl. CPC A61K 31/506; A61K 31/454; A61K 38/08; A61P 21/00; G01N 33/6893; G01N 33/74; G01N 2333/5754; G01N 2800/52; G01N 2800/7052

(57) ABSTRACT

The present invention concerns endothelin receptor antagonists for use in the treatment or the prevention of muscle fibrosis.

The present invention concerns new therapeutic uses of endothelin receptor antagonists.

### BACKGROUND OF THE INVENTION

In adult skeletal muscle, the extracellular matrix (ECM) plays an important role for the contractile muscle fibers. The ECM is a three-dimensional network consisting of extracellular macromolecules, such as collagen and glycoproteins, but also soluble factors such as growth factors and cytokines, to provide structural and biochemical support to the surrounding cells. The ECM regulates bioavailability of biofactors and maintains muscle fiber structure during regeneration. Muscle regeneration is a complex time-regulated process orchestrated by a variety of key cellular actors including satellite cells, muscle fibers, inflammatory, fibroadipogenic progenitors and endothelial cells. During this process ECM undergoes tremendous changes, in term of quantity and quality and all these changes induce specific responses in the different cell types involved in muscle regeneration (Bentzinger et al., 2012). Fibrosis, characterized by excessive ECM accumulation, is a consequence of tissue inflammation or damage and further results in scarring and leads to the impairment of the affected organ. Fibrosis can occur in many tissues of the body, including the skeletal muscle, wherein it is a common pathological hallmark of muscular dystrophies (Smith & Barton, 2018). Fibrosis is probably one of the most detrimental symptoms in muscular dystrophies, which is still not deciphered in humans. Moreover, muscle fibrosis can compromise the efficiency of gene and cell based therapies developed for muscular dystrophies. As yet there is no efficient treatment to reverse this process in skeletal muscle. Such treatments would be essential not only to counteract the pathological process, but also to improve the efficiency of other therapeutic strategies such as gene or cell therapy, largely hampered by the presence of fibrosis. On the other side, the accumulation of extracellular matrix in muscle tissue also affects elderly subjects without any muscular dystrophy, such as elderly subjects suffering from achalasia, for which there is neither efficient drug.

In mouse, fibrosis involves mechanical, humoral, cellular factors and also soluble factors such as TGFβ, which has been described as one of the key drivers of ECM remodeling (Abrigo et al, 2018), pro- and anti-inflammatory cytokines, and growth factors such as CTGF and PDGF (Serrano et al, 2010). At the cellular level, fibrosis involves inflammatory cells, fibro-adipogenic progenitors (FAPs), and possibly satellite cells (Pessina et al, 2015). FAPs are the major collagen-producing cells within the stromal tissue microenvironment and these cells have an increasingly appreciated role as an autocrine source of profibrotic stimuli associated with fibrosis in dystrophic conditions. However, the involvement of FAPs in the evolution of muscle dystrophies in human is still not completely clear, emphasizing the need to better understand the molecular and cellular actors in human fibrosis that is often aggravating muscle dystrophies. Many aspects of the cellular cross talk contributing to the initiation and maintenance of fibrosis still need to be addressed in human skeletal muscle.

Up to date, there is no efficient drug addressed to muscle fibrosis. Thus, it is necessary to provide an efficient strategy for treating muscle fibrosis, either developed in patients suffering from myopathies, in particular muscular dystrophies, or in elderly persons. It is also desirable to develop an efficient drug for use in the treatment or prevention of muscle fibrosis, which improves muscle tissue state and can be further combined with genetic therapies or cellular therapies for treating muscular dystrophies.

Thus, there is a need to provide a new therapy in order to treat or prevent muscle fibrosis.

### SUMMARY OF THE INVENTION

The inventors observed that human FAPs from fibrotic muscles are different as compared with those from healthy non-fibrotic control muscles, showing a strikingly higher proliferative capacity, an exacerbated ECM protein production and the ability to inhibit myoblasts fusion. In order to identify potential candidate proteins expressed by FAPs from fibrotic muscles that could influence muscle differentiation, the inventors have analyzed the transcriptomic profiles of 189 genes deregulated in control human cricopharyngeal fibrotic muscle (CPM) FAPs and identified 111 candidate proteins with a predicted extracellular nature. After multiple series of screening, endothelin receptor B was identified as a particularly interesting candidate target for inhibiting fibrosis progression. Indeed, the inventors observed an upregulation of this receptor in both control fibrotic muscle FAPs and pathological/OPMD (oculopharyngeal muscular dystrophy) fibrotic muscle FAPs. The ligand of endothelin receptor B is endothelin 1 (ET1), which is known as a profibrotic peptide, and is secreted by myotubes (Le Bihan et al., 2012). ET-1 is a widely distributed, multifunctional hormone that operates in both paracrine and autocrine manner. ET-1 is the most potent, long-lasting vasoconstrictor ever discovered in humans (Yanagisawa et al. 1988). Originally described as being secreted by endothelial cells and having an effect on smooth muscle cells, ET-1 has been associated with the development of fibrosis in different organs, including lung, cardiac fibroblasts or hepatic stellate cells (Abraham et al., 1997; Katwa et al., 2003; Rockey et al., 1998). However, the effect of ET-1 on FAPs is completely unknown in the prior art. In addition, endothelin receptor B expression profile in FAPs is never described in the prior art. The present Inventors have shown for the first time that ET-1 stimulates ECM production in fibrotic skeletal muscle FAPs but not in non-fibrotic control skeletal muscle FAPs. Moreover, the Inventors have shown that the addition of an endothelin receptor B antagonist to cells from skeletal fibrotic muscle can decrease ECM production by FAPs and partially restores impaired myotubes fusion. Thus the blockade of the endothelin pathway via its receptors offers a new target for the treatment of muscle fibrosis.

Thus, one aspect of the present invention is to provide an endothelin receptor antagonist for use in the treatment or in the prevention of muscle fibrosis, in particular human muscle fibrosis.

Another aspect of the present invention is related to a pharmaceutical composition comprising a therapeutically effective amount of at least one endothelin receptor antagonist, and a pharmaceutical acceptable carrier, for use in the treatment or prevention of muscle fibrosis.

Another aspect of the present invention is related to a kit-of-parts for simultaneous, separate or sequential use for the treatment or prevention of muscle fibrosis in patients suffering from a myopathy, in particular a muscular dystrophy, said kit comprising at least one endothelin receptor antagonist, and the means for carrying out a gene therapy or cellular therapy or pharmacological therapy for use in the treatment of a myopathy, in particular a muscular dystrophy.

The present invention also provides an in vitro method for diagnosing muscle fibrosis in a human subject suspected of suffering from muscle fibrosis, said method comprising:
determining the circulating endothelin level in a biological sample obtained from said human subject; and
comparing said circulating endothelin level with a reference level to correlate said circulating endothelin level to the presence or absence of muscle fibrosis in said human subject.

The present invention also provides a method for evaluating the effectiveness of a treatment of muscle fibrosis. Said method comprises:
determining the circulating endothelin level in a biological sample obtained from a human subject under a treatment of muscle fibrosis; and
comparing said circulating endothelin level with an initial level to evaluate the effectiveness of said treatment of muscle fibrosis.

### LEGENDS OF THE FIGURES

FIG. 1A: Quantification of ET-1 protein in unconcentrated conditioned medium secreted from differentiated human myoblasts at 24 h, 48 h and 72 h of differentiation.

FIG. 1B: RT-qPCR quantification of EDNR b gene expression normalized to RPLP0 expression in FAPs from control skeletal muscle (M^{CT}), control fibrotic muscle (FibM^{CT}) and pathological/OPMD fibrotic muscle (FibM^{OP}) (**P<0.01).

FIG. 1C: RT-qPCR quantification of human EDNR b gene expression normalized to hB2M expression in mouse Tas injected with FAPs from M^{CT}, FibM^{CT} and FibM^{OP} (***P<0.001****P<0.0001).

FIG. 2A: Immunofluorescence images of FAPs from M^{CT}, FibM^{CT} and FibM^{OP} cultured with DMSO, ET-1 (40 nM)+/−Bosentan (10 μM). Phalloidin (red), Hoechst (blue) and Collagen7a1 (green)

FIG. 2B: Quantification of the percentage of COL7A1 positive cells after a treatment with DMSO or ET-1 (40 nM) or ET-1 and Bosentan (10 μM). COL7A1 is used as a readout of ECM production.

FIG. 2C: Assessment of the proliferation (percentage of Edu positive cells) after a treatment with DMSO or ET-1 (40 nM) or ET-1 and Bosentan (10 μM).

FIG. 3A: Fusion index of myoblast alone, or cocultured with FAPs from FibM^{OP} in the presence or not of Bosentan (10 μM) (*P<0.05, **P<0.01, ****P<0.0001).

FIG. 3B: Immunostaining of Desmin (green) and Hoechst (blue) performed on myoblast alone or cocultured with FAPs from FibM^{OP} in the presence or not of Bosentan (10 μM).

FIG. 4A: Quantification of the percentage of COL7A1 positive cells after a treatment with DMSO or ET-1 (40 nM) or ET-1 and Bosentan (10 μM) or ET-1 and BQ788 (10 nM) or ET-1 and BQ123 (10 nM). COL7A1 is used as a readout of ECM production.

FIG. 4B: Assessment of the proliferation (percentage of Edu positive cells) after a treatment with DMSO or ET-1 (40 nM) or ET-1 and Bosentan (10 μM) or ET-1 and BQ788 (10 nM).

FIG. 5A: RT-qPCR quantification of EDNR b gene expression normalized to RPLP0 expression in FAPs from control skeletal muscle (M^{CT}), control fibrotic muscle (FibM^{CT}), pathological/OPMD fibrotic muscle (FibM^{OP}) and pathological/IBM fibrotic muscle (FibM^{IBM}) (**P<0.01, ****P<0.0001).

FIG. 5B: RT-qPCR quantification of EDNR a gene expression normalized to RPLP0 expression in FAPs from control skeletal muscle (M^{CT}), control cricopharyngeal fibrotic muscle (FibM^{CT}) pathological/OPMD fibrotic muscle (FibM^{OP}) and pathological/IBM fibrotic muscle (FibM^{IBM}).

### DETAILED DESCRIPTION OF THE INVENTION

The inventors have conducted experiments on FAPs derived from skeletal muscles to identify key regulators of skeletal muscle fibrosis. They have surprisingly shown that the endothelin receptor is such a key regulator. Therefore, a first aspect of the present invention is related to an endothelin receptor antagonist for use in the treatment or the prevention of muscle fibrosis.

The term “endothelin receptor” herein refers to all endothelin receptors, in particular endothelin receptor type A (ET_{A}), endothelin receptor type B (ET_{B}), or any endothelin receptor-like G-protein-coupled receptors which can bind with an endothelin, in particular an isoform of human endothelin, more particularly endothelin-1, endothelin-2, or endothelin-3.

Within the scope of the present invention, the terms “endothelin receptor type A”, “endothelin type-A receptor”, “endothelin receptor A”, “ET_{A} receptor” and “ENDRA” are interchangeable.

Within the scope of the present invention, the terms the terms “endothelin receptor type B”, “endothelin type-B receptor”, “endothelin receptor B”, “ET_{B} receptor” and “ENDRB” are interchangeable

The term “endothelin receptor antagonist” herein refers to any substance which can block or reduce the binding between an endothelin and its endothelin receptor and thus stops or reduces the in vivo activation of said endothelin receptor.

In a particular embodiment, said endothelin receptor antagonist is an antagonist of a human endothelin receptor.

In another particular embodiment, said endothelin receptor antagonist is an antagonist of ET_{A} receptor, in particular an antagonist of human ET_{A} receptor, or an antagonist of ET_{B} receptor, in particular an antagonist of human ET_{B} receptor.

In a particular embodiment, said endothelin receptor antagonist is a substance which can block or reduce the binding between endothelin-1 with a human endothelin receptor, in particular human endothelin receptor B (ENDRB) or human endothelin receptor A (ENDRA).

Said endothelin receptor antagonist can be a chemical molecule, a protein, a fragment of a protein, a peptide, or an aptamer, which competes with any isoform of endothelin, in particular an isoform of human endothelin, in particular endothelin-1, for the binding to endothelin receptor.

According to an embodiment of the invention, said endothelin receptor antagonist can be a dual endothelin receptor antagonist, an antagonist which binds selectively to endothelin type-B receptor, or an antagonist which binds selectively to endothelin type-A receptor.

As used therein, the term “dual endothelin receptor antagonist” refers to an antagonist which blocks both ET_{A} and ET_{B} receptors, in particular human ET_{A} and ET_{B} receptors. Examples of such dual endothelin receptor antagonist include Bosentan, macitentan, aprocitentan, or tezosentan.

As used herein, the term “antagonist which binds selectively to endothelin type-A receptor” refers to an antagonist which has a greater affinity with endothelin type-A receptor than with endothelin type-B receptor, in particular an antagonist which has a greater affinity with human ET_{A} receptor than with human ET_{B} receptor. Examples of such antagonists are BQ123, sitaxsentan, atrasentan, avosentan, ambrisentan, an antibody directed to an ET_{A} receptor, an antibody mimetic directed to an ET_{A} receptor and an aptamer directed to an ET_{A} receptor.

As used herein, the term “antagonist which binds selectively to endothelin type-B receptor” refers to an antagonist which has a greater affinity with endothelin type-B receptor than with endothelin type-A receptor, in particular an antagonist which has a greater affinity with human ET_{B} receptor than with human ET_{A} receptor. Examples of such antagonists are IRL2500, K-8794, RES7011, Ro 46-8443, A192621, BQ788, an antibody directed to an ET_{B} receptor, an antibody mimetic directed to an ET_{B} receptor and an aptamer directed to an ET_{B} receptor.

An antagonist of an endothelin receptor for use according to the present invention can be an antibody directed to said receptor or an antibody mimetic which specifically binds with an endothelin receptor. Antibodies used in the present invention as antagonists of ET_{A} or ET_{B} receptor, in particular human ET_{A} or ET_{B} receptor, include but are not limited to monoclonal or polyclonal antibodies, chimeric antibodies, humanized antibodies, fully human antibodies, and nano-antibodies, full length or fragments thereof including Fab, Fab′ or F(ab′)₂, scFV, or diabody. An antibody mimetic for use in the present invention can be a peptide designed by any conventional methods, particularly with the help of routinely used bioinformatic software, basically according to the analysis of the binding domains between the ET_{A}/ET_{B} receptor and its neutralizing antibodies. Antibodies or antibody mimetics for use in the present invention bind to targeted endothelin receptor in competition with endothelin, in particular ET-1.

An antagonist of endothelin receptor for use according to the present invention can also be an aptamer directed to said receptor. Said aptamer can be selected by standard methods, for example from a large oligonucleotide library through SELEX (Sequential Evolution of Ligands by Exponential Enrichment) process. Aptamers used in the present invention bind to targeted endothelin receptor in competition with endothelin, in particular ET-1.

According to a particular embodiment, the endothelin receptor antagonist used in the present invention is an antagonist which binds to human ET_{B} receptor. Illustrative endothelin receptor antagonists of the human ET_{B} receptor include dual endothelin receptor antagonists, antagonists which bind selectively to human ET_{B} receptor, antibodies or antibody mimetics directed to human ET_{B} receptor and aptamers directed to human ET_{B} receptor.

According to another particular embodiment, the endothelin receptor antagonist used in the present invention is an antagonist which binds to human ET_{A} receptors. Illustrative endothelin receptor antagonists of the human ET_{A} receptor include dual endothelin receptor antagonists, antagonists which bind selectively to endothelin type-A receptor, antibodies or antibody mimetics directed to human ET_{A} receptor and aptamers directed to human ET_{A} receptor.

In a preferred embodiment, said endothelin receptor antagonist is selected from Bosentan, TAK044, SB209670, A192621, BQ788, IRL2500, atrasentan, K-8794, RES7011, Ro 46-8443, macitentan, aprocitentan, ambrisentan, BQ123, sitaxsentan, an antibody, an antibody mimetic or an aptamer directed to human endothelin type-A receptor and an antibody, an antibody mimetic or aptamer directed to human endothelin type-B receptor.

In a more preferred embodiment, said endothelin receptor antagonist is selected from Bosentan, macitentan, aprocitentan, or tezosentan, IRL2500, K-8794, RES7011, Ro 46-8443, A192621, BQ788, an antibody directed to an ET_{B} receptor, an antibody mimetic directed to an ET_{B} receptor and an aptamer directed to an ET_{B} receptor.

In another preferred embodiment, said endothelin receptor antagonist is selected from BQ123, Bosentan or BQ788, more particularly Bosentan or BQ788, still more particularly Bosentan.

In the context of the present invention, above-described endothelin receptor antagonists are used in the treatment or in the prevention of muscle fibrosis. Indeed, as shown in the examples, the inventors have shown thanks to experiments conducted on FAPs derived from skeletal muscles that fibrosis of skeletal muscles can be controlled with endothelin receptor antagonists.

Muscle fibrosis is characterized by excessive accumulation of extracellular matrix components. and is most readily developed in patients suffering from myopathies, in particular muscular dystrophies. However, muscle fibrosis can also be observed in patients without myopathies, such as in elder subjects. By the way, muscle trauma can also be a cause of muscle fibrosis. Clinically, muscle fibrosis can be diagnosed by histological analysis of biopsies and/or by non-invasive imaging techniques, such as magnetic resonance imaging (MRI).

According to an embodiment, the present invention is related to the above-described endothelin receptor antagonist, in particular antagonists which binds to human ET_{B} receptor, for use in the treatment or the prevention of muscle fibrosis developed in patients suffering from myopathies, in particular muscular dystrophies, such as oculopharyngeal muscular dystrophy, Duchenne muscular dystrophy, facioscapulohumeral muscular dystrophy, Becker muscular dystrophy, Limb-girdle muscular dystrophy, Distal muscular dystrophy, congenital muscular dystrophy, or Emery-Dreifuss muscular dystrophy, or other forms of myopathies, such as inclusion body myositis.

According to another embodiment, the present invention is related to the above-described endothelin receptor antagonist, in particular antagonists which binds to human ET_{B} receptor, for use in the treatment or the prevention of muscle fibrosis developed in patients without myopathy, such as muscle fibrosis developed in patient without myopathy but suffering from dysphagia or in an elderly person without myopathy but suffering from achalasia.

In a particular embodiment, the present invention is related to an endothelin receptor antagonist for use in the treatment or the prevention of pharyngeal muscle fibrosis, such as that developed in patients suffering from oculopharyngeal muscular dystrophy or inclusion myositis, or in elderly persons suffering from achalasia.

The term “treatment” or “treating” includes administering to a subject an effective amount of an endothelin receptor antagonist to inhibit, reduce or reverse the progression of a disease, condition or disorder, or ameliorate clinical symptoms of a disease. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

A treatment can be considered as effective, if after a determined period, a statistically significant difference can be observed for any one of above four criteria between a treated patient and mean value calculated from non-treated patients or between the conditions of said patient before the treatment and after the treatment. The existence or not of said difference can be determined by any medical analysis method routinely used by physicians for evaluating the progression of a disease, particularly any conventional method for evaluating the progression of muscular fibrosis, for example by histological and biochemical assays or by NMR and ultrasound as described by Martin-Bach et al. 2021.

The term “prevention” or “preventing” includes administering to a subject an effective amount of an endothelin receptor antagonist to prevent the appearance of clinical symptoms characteristic of a disease.

A prevention can be considered as effective, if after a determined period, the development or the appearance of symptoms characteristic of the disorder(s) in a treated patient is delayed compared to mean value obtained from non-treated patients.

According to another particular embodiment, circulating endothelin level measured in a biological sample, in particular a blood sample, can also be used for determining the effectiveness of an antagonist of endothelin receptor for use according to the present invention. According to said embodiment, the circulating endothelin level measured in a subject under the treatment of said antagonist is compared with that measured in said subject before the treatment. A decrease of circulating endothelin level in a subject under treatment is an indicator of the effectiveness of said antagonist for treating muscle fibrosis, while an equal or increase of circulating endothelin level in a subject under treatment is an indicator of the ineffectiveness of said antagonist for treating muscle fibrosis.

According to still another particular embodiment, the effectiveness of an antagonist of endothelin receptor for use according to the present invention can also be determined by comparing the circulating endothelin level measured in a subject under the treatment of said antagonist with a reference level established from healthy subject.

The circulating endothelin level, in particular the circulating endothelin-1 level, in a biological sample can be measured by any conventional methods, such as by an immunoassay or by UPLC-MS/MS (Suzuki et al., J Pharm Biomed Anal. 2017 Aug. 5; 142:84-90.) The present invention also provides a pharmaceutical composition comprising aforementioned endothelin receptor antagonist for use in the treatment or prevention of muscle fibrosis. Such composition comprises a therapeutically effective amount of at least one endothelin receptor antagonist, and a pharmaceutical acceptable carrier.

The term “pharmaceutically acceptable carrier” means a carrier that is useful in preparing a pharmaceutical composition or formulation that is generally safe, non-toxic, and neither biologically nor otherwise undesirable, and includes a carrier that is acceptable for human pharmaceutical use. The carrier can act as a vehicle, medium, or for dilution of the active ingredient. The formulation of the pharmaceutical composition of the present invention can be determined and carried out according to well-known prior art relating to drug formulation. The carrier material can be an organic or inorganic inert carrier material, for example one that is suitable for oral administration or injection. Suitable carriers include water, gelatin, arabic gum, lactose, starch, magnesium stearate, talc, animal or vegetable oils, polyalkylene-glycols, glycerine and petroleum jelly. The composition may also comprise additional additives such as flavoring agents, preservatives, stabilizers, wetting agents, emulsifiers, and/or salts for varying the osmotic pressure and/or buffers.

The compositions can be formulated in any conventional form including a solid form for oral administration such as tablets, pills, powders, lozenges, sachets, cachets, elixirs, suspensions, emulsions, solutions, syrups, soft and hard gelatin capsules, suppositories, sterile injectable solutions, and sterile packaged powders.

The compositions of the invention can also be administered to a patient in accordance with the invention by topical (including transdermal, buccal or sublingual), or parenteral (including intraperitoneal, subcutaneous, intravascular (i.e. intravenous or intra-arterial), intradermal or intramuscular injection) routes. Suitable administration route can be determined by a skilled person according to the location and/or the origin of muscle fibrosis. For instance, the composition of the invention for the treatment of oculopharyngeal muscular dystrophy (OPMD) is preferably administrated by oral administration.

The term “therapeutically effective amount” means the amount of an aforementioned endothelin receptor antagonist as pharmaceutical active in a pharmaceutical composition to produce the desired therapeutic effect.

For example, the “therapeutically effective amount” of an aforementioned endothelin receptor antagonist for use according to the present invention includes, but is not limited to, an amount of 50-1000 mg/day, 50-900 mg/day, 50-800 mg/day, 50-700 mg/day, 50-600 mg/day, 50-500 mg/day, particularly of 50-400 mg/day, 50-300 mg/day, 60-300 mg/day, 70-300 mg/day, 80-300 mg/day, 60-250 mg/day, more particularly of 80-250 mg/day.

Particularly, the “therapeutically effective amount” of Bosentan for use according to the present invention includes, but is not limited to, an amount of 50-500 mg/day, particularly from 50-400 mg/day, 50-300 mg/day, 60-250 mg/day, more particularly from 80-250 mg/day.

A “therapeutically effective amount” of an aforementioned endothelin receptor antagonist for use according to the present invention can be determined by standard techniques, for example by in vivo and/or in vitro assays. A skilled person can also determine the optimal dosage ranges or adapt the dosage according to administration route and frequency, the age, weight, sex, medical condition of the patient, the severity of the disease, and/or the therapeutic objective of the treatment or prevention.

The present invention also provides a method for treating or preventing muscle fibrosis in a subject in need thereof, comprising administering to said subject a therapeutically effective amount of an endothelin receptor antagonist as described before.

In another aspect of the invention, an endothelin receptor antagonist as described above can be used in combination with a gene therapy or cellular therapy for treating myopathy, in particular muscular dystrophy. Indeed, muscle fibrosis may be a hindrance to the performance and the efficiency of a such gene therapy or cellular therapy. As shown by the present invention, an endothelin receptor antagonist allows to treat or prevent muscle fibrosis, this in turn allows a gene therapy or cellular therapy to more efficiently reach target tissue or cells and accordingly to provide the maximal efficiency of treatment.

Thus, the present invention also provides a kit-of-parts for simultaneous, separate or sequential use in the treatment or the prevention of muscle fibrosis in patients suffering from a myopathy, in particular a muscular dystrophy. Said kit comprises or consists of:
at least one endothelin receptor antagonist, and
the means for carrying out a gene therapy or cellular therapy or pharmacological therapy for use in the treatment of a myopathy, in particular a muscular dystrophy.

Said gene therapy can be for example an adeno-associated virus-based gene therapy for treating oculopharyngeal muscular dystrophy (Malerba et al., 2017).

Said cellular therapy can be for example the means for autologous myoblast transplantation (Périé et al., 2014).

Said pharmacological therapy can be for example an anti-aggregation drug, in particular Guanabenz, for treating oculopharyngeal muscular dystrophy (Malerba et al., 2019) The means for carrying out such gene therapy or cellular therapy can be for example viral vectors, in particular AAV vectors, for performing a gene therapy or cells suitable for performing a cellular therapy. Said vectors can harbor antisense oligonucleotides, shRNA, or oligonucleotides encoding functional human proteins. Said cells suitable for cellular therapy can be for example autologous or heterologous tissue cells, pluripotent or multipotent stem cells from the patient himself or from a donor.

The treatment or the prevention of muscle fibrosis by an endothelin receptor antagonist as described above can be performed before, simultaneously or after a gene therapy or cellular therapy.

Said endothelin receptor antagonist contained in a kit-of-parts of the present invention can be any aforementioned antagonist, in particular an antagonist of ET_{B} receptor or an antagonist of ET_{A} receptor or a combination of an antagonist of ET_{B} receptor and an antagonist of ET_{A} receptor. Said endothelin receptor antagonist can be in particular a dual endothelin receptor antagonist, an antagonist which binds selectively to endothelin type-B receptor, or an antagonist which binds selectively to endothelin type-A receptor, in particular an endothelin receptor antagonist selected from Bosentan, TAK044, SB209670, A192621, BQ788, IRL2500, atrasentan, K-8794, RES7011, Ro 46-8443, macitentan, aprocitentan, ambrisentan, BQ123, sitaxsentan, an antibody, an antibody mimetic or an aptamer directed to human endothelin type-A receptor and an antibody, an antibody mimetic or aptamer directed to human endothelin type-B receptor. In a more particular embodiment, said endothelin receptor antagonist is Bosentan, BQ123 or BQ788.

Another aspect of the present invention is to provide an in vitro method for diagnosing muscle fibrosis in a human subject suspected of suffering from muscle fibrosis.

Said method comprises:
determining the circulating endothelin level in a biological sample obtained from said human subject; and
comparing said circulating endothelin level with a reference level to correlate said circulating endothelin level to the presence or absence of muscle fibrosis in said human subject.

The term “a human subject suspected of suffering from muscle fibrosis” refers to a human subject who suffers from one of the diseases which are generally known as causes of muscle fibrosis, such as muscle dystrophies or a subject who has experienced trauma to muscle which can also be the cause of muscle fibrosis. A human subject suspected of suffering from muscle fibrosis is a person for whom there is serious doubt that muscle fibrosis may develop compared to all others pathologies which may also lead to a variation of circulating endothelin level.

The term “biological sample” refers to any biological sample which can be obtained from a human subject, in particular a sample of body fluids, such as urine or blood. In a preferred embodiment of the present invention, the biological sample is a blood, plasma or serum sample.

The term “circulating endothelin” refers to an isoform of secreted endothelin. Particularly, said circulating endothelin is endothelin-1, endothelin-2, or endothelin-3. Said circulating endothelin may be present in a biological sample, in particular in body fluids, more in particular in blood, plasma or serum. In a particular embodiment, said circulating endothelin is circulating endothelin-1 (ET1).

According to the diagnostic method of the present invention, the circulating endothelin level, in particular the circulating endothelin-1 level, in a biological sample can be measured by any conventional methods, such as by an immunoassay or by UPLC-MS/MS (Suzuki et al., J Pharm Biomed Anal. 2017 Aug. 5; 142:84-90.)

According to a particular embodiment, the reference level used in the diagnostic method of the present invention is a mean value of circulating endothelin level calculated from a group of healthy human subjects. Further criterion such as age, sex, other physical or healthy conditions, or genetic background can also be taken into account for constituting said group.

In the context of the diagnostic method of the present invention, an increase of circulating endothelin level measured in said human subject suspected of suffering from muscle fibrosis compared to the reference level is indicative of the potential presence of muscle fibrosis.

In some embodiments, when circulating endothelin level in a biological sample of a human subject suspected of suffering from muscle fibrosis is at least 10%, 20%, 30%, 40%, 50%, 60%, 70%, 80%, 90%, 100%, 150%, 200%, 250%, or 300% higher than a reference level, it is considered that there is an increase of circulating endothelin level in said human subject and there is probability that said human subject is suffering from a muscle fibrosis.

According to an embodiment, the present invention also provides a method for monitoring the evolution of a muscle fibrosis in a human subject suspected of suffering from muscle fibrosis. Said method comprises:
determining the circulating endothelin level in a biological sample obtained from said human subject; and
comparing said circulating endothelin level with a previous level measured in a biological sample obtained from the same human subject.

In the context of this method, a “previous level” is referred to the level measured in a biological sample obtained from the same human subject before a determined period of time. According to this method, an increase of circulating endothelin level in said human subject compared to the previous level is an indicator of the progression of muscle fibrosis; a reduction of circulating endothelin level in said human subject compared to the previous level is an indicator of a diminution of muscle fibrosis.

The present invention also provides a method for evaluating the effectiveness of a treatment of muscle fibrosis. Said method comprises:
determining the circulating endothelin level in a biological sample obtained from a human subject under a treatment of muscle fibrosis; and
comparing said circulating endothelin level with an initial level to evaluate the effectiveness of said treatment of muscle fibrosis.

In the context of this method, an “initial level” is referred to the level measured in a biological sample obtained from the same human subject before a treatment of muscle fibrosis. Accordingly, a decrease of circulating endothelin level in said human subject compared to the initial level is an indicator of the effectiveness of said treatment; an increase or equal level of circulating endothelin in said human subject compared to the initial level is an indicator of inefficient treatment tor less efficient treatment.

The present invention is illustrated in more detail by following examples.

### Examples

### 1. Materials and Methods

### Cell Cultures

Human FAPs cells were isolated from muscle biopsies obtained during surgical procedures via Myobank, affiliated to EuroBioBank, after informed consent in accordance with European recommendations and French legislation (authorization AC-2019-3502). Human FAPs isolated here as the CD56 negative cell fraction (Périé, S. et al. 2006), are obtained from control skeletal muscle: M^{CT}, control fibrotic cricopharyngeal muscle (CPM): FibM^{CT}, OPMD fibrotic CPM with exacerbated levels of fibrosis: FibM^{OP}, and fibrotic muscle from inclusion body myositis patients: FibM^{IBM}. All tested fibrotic muscle are skeletal fibrotic muscle. These cells expressed predominantly PDGFRa, CD90 and CD105 known markers of Fibro/Adipogenic Progenitors (FAPs) in human skeletal muscle. The tissue type, cell type, age, pathological condition and muscle fibrotic state of each biopsy sample are specified in table below.

| Biopsy | Cell type | years old | Dystrophy | Fibrotic muscle |
|---|---|---|---|---|
| M^{CT} | FAPS | 95 | — | no |
| M^{CT} | FAPS | 89 | — | no |
| M^{CT} | FAPS | 39 | — | no |
| M^{CT} | FAPS | 61 | — | no |
| M^{CT} | FAPS | 88 | — | no |
| M^{CT} | FAPS | 51 | — | no |
| FibM^{CT} | FAPS | 52 | — | yes |
| FibM^{CT} | FAPS | 88 | — | yes |
| FibM^{CT} | FAPS | 58 | — | yes |
| FibM^{CT} | FAPS | 92 | — | yes |
| FibM^{CT} | FAPS | 86 | — | yes |
| FibM^{OP} | FAPS | 73 | OPMD | yes |
| FibM^{OP} | FAPS | 63 | OPMD | yes |
| FibM^{OP} | FAPS | 78 | OPMD | yes |
| FibM^{OP} | FAPS | 60 | OPMD | yes |
| FibM^{OP} | FAPS | 62 | OPMD | yes |
| FibM^{OP} | FAPS | 67 | OPMD | yes |
| FibM^{OP} | FAPS | 72 | OPMD | yes |
| FibM^{OP} | FAPS | 78 | OPMD | yes |
| FibM^{OP} | FAPS | 78 | OPMD | yes |
| FibM^{IBM} | FAPS | 65 | IBM | yes |
| M^{CT} | myoblasts | 20 | — | No |

Muscle fibrotic state of each biopsy sample is determined by histological analysis according to conventional methods.

The muscle biopsies were finely minced and explants were plated onto non-coated Petri dishes in drops of fetal bovine serum (FBS) (Invitrogen, Carlsbad, CA) as previously described (Bigot et al., 2009). Cells were cultivated at 37° C. in a humid atmosphere containing 5% CO₂ and in growth medium consisting of 199 medium (Life technologies, Paisley, UK) and Dulbecco's Modified Eagle Medium (DMEM, Life technologies) in a 1:4 ratio supplemented with 20% Fetal Bovine Serum (FBS, Invitrogen), 25 μg/ml fetuin (Life technologies), 0.5 ng/mL bFGF (Life technologies), 5 ng/ml EGF (Life technologies), 5 μg/mL insulin (Sigma-Aldrich) and 50 μg/mL gentamycin (Life Technologies). Cells were labelled with CD56 antibody coupled to microbeads (130-050-401, MACS; Miltenyi Biotec, Paris, France) and then separated using a immunomagnetic cell sorting system (MACS) according to the manufacturer's instructions. The myogenic purity of both cell fractions (CD56+, myogenic and CD56−, non-myogenic) was monitored by immunocytochemistry using an antibody against desmin (clone D33, Dako, Trappes, France), exclusively expressed in myogenic cells.

### Animals

2-3 month old Rag2−/−Il2rb−/− immunodeficient mice were used as recipients for human cells transplantation. Mice were anaesthetized by an intraperitoneal injection of 80 mg/kg of ketamine hydrochloride and 10 mg/kg xylazine (Sigma-Aldrich. St. Louis, MO). This study was carried out in strict accordance with the legal regulations in France and according to European Union ethical guidelines for animal research. The protocol was approved by the Committee on the Ethics of Animal Experiments Charles Darwin No 5 (Protocol Number: 02704.01). All surgery was performed under ketamine hydrochloride and xylasine anestesia, and all efforts were made to minimize suffering.

### Cell Transplantation

Cultures of human cells were washed in PBS, trypsinized, centrifuged, and re-suspended in PBS. The cells were injected into both Tibialis Anterior (TA) muscles. Prior to injection, the Tas of the immunodeficient mice were subjected to three freeze lesion cycles of ten seconds each in order to damage the muscle fibers, and trigger regeneration. Cultures of human cells were implanted into the recipient's muscle immediately after cryodamage, using a 25 μl Hamilton syringe as previously described (Negroni et al., 2009). For CD56− cells, 15 μl of cell suspension containing 1.4×10⁵ cells in PBS were injected immediately after cryodamage, then 4 and 8 days after. Mice were sacrificed 1 month after the first injection and the Tas were collected and stored at −80° C. for analysis.

### Coculture Experiments

Non-myogenic (CD56−) and myogenic (CD56+) cells were seeded together at a 30%/70% ratio and at a final confluence of 21000 cells/cm2. Once the cells have adhered, the medium was replaced by a differentiation medium composed by DMEM with 50 μg/mL gentamycin. To study the effect of Bosentan on the fusion index in coculture 10 μM of Bosentan (SML1265, Sigma Aldrich) was added to the wells at day 0 and day 3. Cells were fixed at day 5 in 4% paraformaldehyde (PFA). The fusion index was calculated as the ratio between the number of nuclei per myotube (>2 nuclei) identified by a desmin staining and the total number of desmin+nuclei.

### ET-1 Experiment

The method for quantification of ET-1 protein is described in Le Bihan et al. 2012. Treatment of FAPs to evaluate the effect of ET-1 was performed on 70-80% confluence cells that were rinsed twice with DMEM and treated 3 days with proliferation medium at 1% FBS (instead of 20%) containing either DMSO or 40 nM ET-1 (E7764, Sigma-Aldrich)+/−10 μM Bosentan (SML1265, Sigma Aldrich), 10 nM BQ788 (SML192621, Sigma Aldrich), or 10 nM BQ123 (B150, Sigma-Aldrich). For proliferation experiment, Edu (10 μM) was added at day 2 and cells were fixed 24 h later. EdU labeling was carried out using the Click-iT™ EdU Cell Proliferation Kit (C10338, Life Technologies) according to the manufacturer's instructions.

### Immunofluorescence

Immunostaining were performed on cells fixed in 4% PFA for 10 minutes (min) and incubated with blocking solution (PBS 2% FBS 0.2% Triton) for 30 minutes (min). Fixed cells were then incubated 1 h with primary antibodies (COL7A1 C6805 Sigma Aldrich 1/800; Desmin M0760 Dako 1/50). Detection of immune complexes was performed using the appropriate Alexa-Fluor-secondary antibodies purchased from Life Technologies (Grand Island, NY) for 45 min. Nuclei and actin filament were counterstained with Hoechst and Phalloidin-Alexa 568 (Interchim 1/400) respectively.

### RNA Extraction and Reverse Transcription

RNA from frozen muscle sections or cell pellets was extracted using TRIzol reagent (Invitrogen, 15596026) according to the manufacturer's instructions. The concentration of RNA was determined with a NanoDrop® spectrophotometer ND-1000. RNA was reverse transcribed using M-MLV (Invitrogen) according to the manufacturer's instructions.

### Quantitative-PCR

Quantitative polymerase chain reaction (qPCR) was carried out using SYBR green mix buffer (Roche Applied Science, Meylan, France) in a LightCycler 480 Real-Time PCR System (Roche Applied Science) as follows: 8 min at 95° C. followed by 50 cycles at 95° C. for 15 seconds (s), 60° C. for 15 s and 72° C. for 15 s, with the program ending in 5 s at 95° C. and 1 min at 65° C. Specificity of the PCR product was checked by melting curve analysis using the following program: 65° C. increasing by 0.11° C./second to 97° C. The gene expression levels were normalized to RPLP0 or hB2M expression and primer sequences are available upon request.

### Statistical Analysis

Data were expressed as the mean±SD. All statistical analyses were performed using GraphPad Prism (version 6.0d, GraphPad Software Inc., San Diego, CA). Statistical significance was assessed by one-way ANOVA test. A difference was considered to be significant at *P<0.05, **P<0.01, ***P<0.001 and ****P<0.0001.

### Experimental Results

### Endothelin Receptor as a Key Regulator in Muscle Fibrosis

To identify potential candidate proteins expressed by FAPs from fibrotic muscles that could influence muscle differentiation, their transcriptomic profiles were analyzed. 189 genes deregulated in the FibM^{CT} FAPs were subjected to stringent computational filtering to predict their cellular localization and identify proteins which were most likely in the extracellular space (Zhao et al., 2019). 111 candidate proteins were identified with a predicted extracellular nature (signal peptide, transmembrane and unconventional secretory pathway).

Among these 111, 66 were upregulated in FibM^{CT} FAPs. The same analysis performed on FibM^{OP} FAPs allowed to identify similarly 63 candidate proteins upregulated in FibM^{OP} FAPs. By combining these two lists, 19 extracellular protein candidates were obtained.

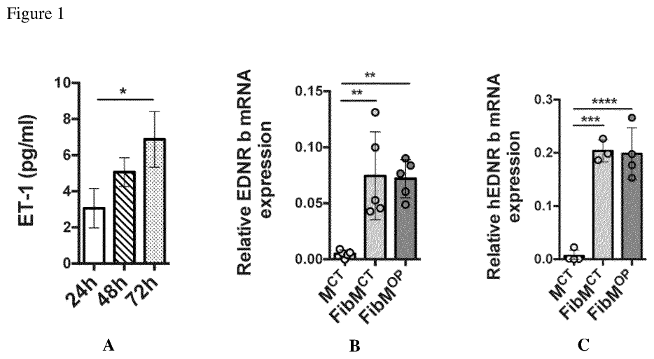

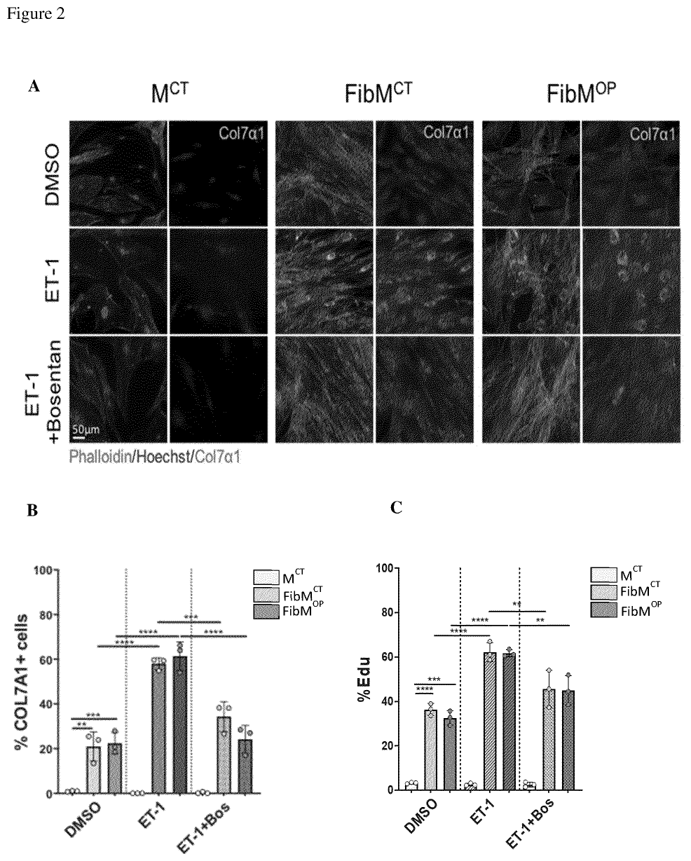

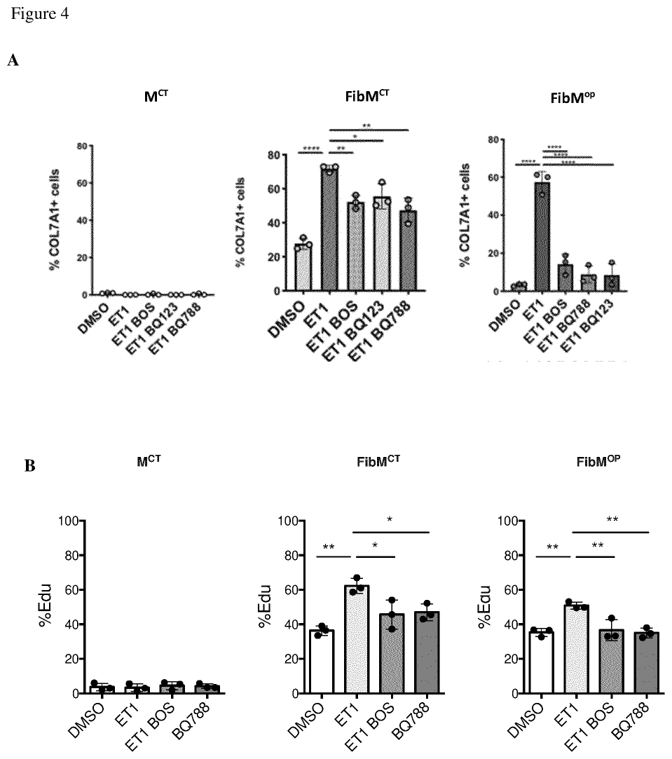

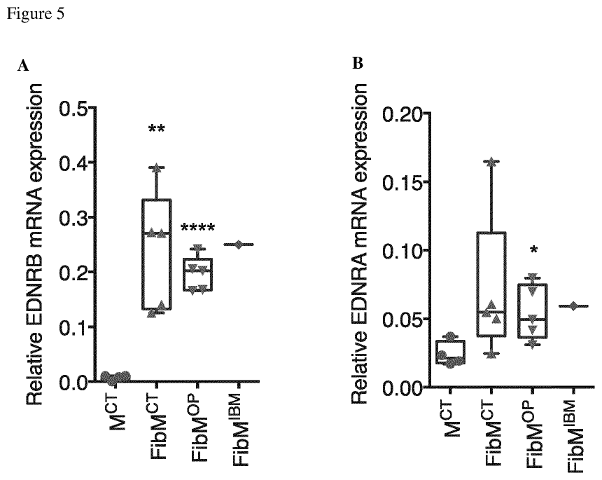

Among the different cellular receptors that were upregulated, ENDRB was identified as a particularly interesting candidate since its ligand: the profibrotic peptide endothelin (ET1) is secreted by myotubes (FIG. 1A). An overexpression of EDNRB in FibM^{CT} and FibM^{OP} FAPs was confirmed by qPCR both in vitro (FIG. 1B) and in vivo (FIG. 1C) when injected in Rag2−/−Il2rb−/− immunodeficient mice. The expression level of EDNRB mRNA was also measured in FibM^{IBM} FAPs. Similarly, we can observe an overexpression of EDNRB mRNA in FibM^{IBM} FAPs compared to its level in M^{CT} (FIG. 5A). The inventors further measured the expression level of EDNRA mRNA in FAPs of M^{CT}, FibM^{CT}, FibM^{OP} and FibM^{IBM}. A significant increase of EDNRA mRNA expression was also observed in FibM^{OP} and FibM^{IB}M FAPs compared to the expression level in M^{CT} (FIG. 5B). Using COL7A1 immunostaining as a readout of ECM production, and Edu to assess proliferation, it was confirmed that addition of ET1 to the culture medium of FAPs increased ECM production and proliferation in FibM^{CT} and FibM^{OP} FAPs but not in M^{C}T FAPs (FIGS. 2A, 2B and 2C). The addition of Bosentan, an ENDR antagonist (Clozel et al. 1994), partially abolished the effect of ET1 (FIGS. 2A, 2B and 2C). Two other ENDR antagonists, BQ788 and BQ123, which are a selective EDNR_{B} antagonist and EDNR_{A} antagonist respectively, were also assessed. The addition of BQ788 and BQ123 also led to the decrease of ECM production in FibM^{CT} and FibM^{OP} FAPs (FIG. 4A). In addition, a decrease of proliferation of FibM^{CT} and FibM^{OP} FAPs after the treatment by BQ788 and Bosentan can also be observed (FIG. 4B). These results illustrate that the blockade of endothelin receptor can reduce secretion and proliferation of FAPs.

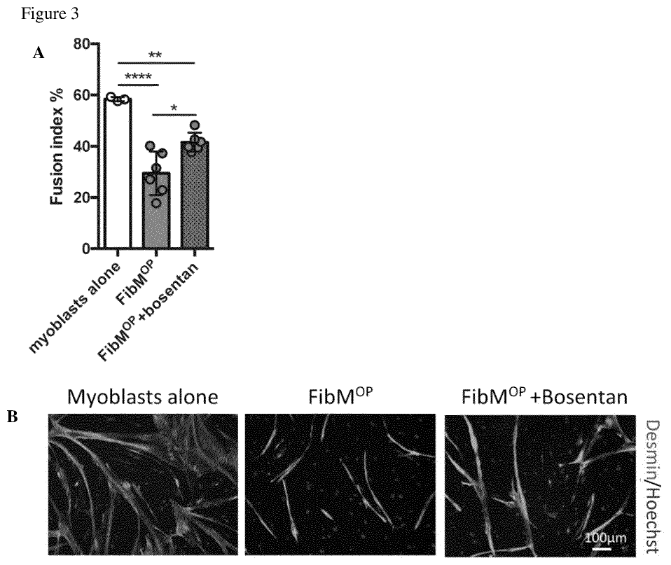

Co-culture of myoblasts with FAPs from fibrotic muscle at 70%/30% ratio impairs myoblasts fusion. In the presence of Bosentan, the fusion index was partially restored to control levels by blocking the endothelin receptor EDNR (FIG. 3A, 3B). Altogether these data demonstrate a key role for endothelin in fibrosis by acting on FAPs in fibrotic tissues.

In conclusion, the data presented above demonstrate the key role of the endothelin receptors of FAPs in fibrosis of human skeletal muscle and identify the endothelin receptor as a new druggable target to counteract human muscle fibrosis.

### REFERENCES

Abraham et al., Increased levels of endothelin-1 and differential endothelin type A and B receptor expression in scleroderma-associated fibrotic lung disease. Am J Pathol, 151 (1997), pp. 831-841
Abrigo, J. et al. Central Role of Transforming Growth Factor Type Beta 1 in Skeletal Muscle Dysfunctions: An Update on Therapeutic Strategies. Curr. Protein Pept. Sci. 19, 1189-1200 (2018).
Bentzinger, C. F., Wang, Y. X. & Rudnicki, M. A. Building Muscle: Molecular Regulation of Myogenesis. Cold Spring Harb Perspect Biol 4, (2012).
Bigot, A. et al. Large CTG Repeats Trigger p16-Dependent Premature Senescence in Myotonic Dystrophy Type 1 Muscle Precursor Cells. The American Journal of Pathology 174, 1435-1442 (2009).
Clozel, M. et al. Pharmacological Characterization of Bosentan, a New Potent Orally Active Nonpeptide Endothelin Receptor Antagonist. J Pharmacol Exp Ther. 1994 July; 270(1):228-35.
Katwa et al., Cardiac myofibroblasts isolated from the site of myocardial infarction express endothelin de novo. Am J Physiol Heart Circ Physiol, 285 (2003), pp. H1132-H1139;
Le Bihan, M.-C. et al. In-depth analysis of the secretome identifies three major independent secretory pathways in differentiating human myoblasts. Journal of Proteomics 77, 344-356 (2012).
Malerba et al., PABPN1 gene therapy for oculopharyngeal muscular dystrophy. Nat Commun. 2017 Mar. 31; 8:14848.
Malerba et al., Pharmacological modulation of the ER stress response ameliorates oculopharyngeal muscular dystrophy. Hum Mol Gene 2019 May 15; 28(10):1694-1708
Martin-Bach et al., Non-invasive assessment of skeletal muscle fibrosis in mice using nuclear magnetic resonance imaging and ultrasound shear wave elastography. Scientific Reports, 2021, volume 11, Article number: 284.
Negroni, E. et al. In Vivo Myogenic Potential of Human CD133+ Muscle derived Stem Cells: A Quantitative Study. Molecular Therapy 17, 1771-1778 (2009).
Pessina, P. et al. Fibrogenic Cell Plasticity Blunts Tissue Regeneration and Aggravates Muscular Dystrophy. Stem Cell Reports 4, 1046-1060 (2015).
Périé, S. et al. Premature proliferative arrest of cricopharyngeal myoblasts in oculo-pharyngeal muscular dystrophy: Therapeutic perspectives of autologous myoblast transplantation. Neuromuscul. Disord. 16, 770-781 (2006).
Périé, S. et al. Autologous Myoblast Transplantation for Oculopharyngeal Muscular Dystrophy: a Phase I/Iia Clinical Study. Mol Ther. 2014 January; 22(1): 219-225.
Smith, L. R. & Barton, E. R. Regulation of fibrosis in muscular dystrophy. Matrix Biology 68-69, 602-615 (2018).
Serrano, A. L. & Munoz-Canoves, P. Regulation and dysregulation of fibrosis in skeletal muscle. Experimental Cell Research 316, 3050-3058 (2010).
Suzuki et al., Ultra-sensitive and selective quantification of endothelin-1 in human plasma using ultra-performance liquid chromatography coupled to tandem mass spectrometry. J Pharm Biomed Anal. 2017 Aug. 5; 142:84-90
Rockey et al. Cellular localization of endothelin-1 and increased production in liver injury in the rat: Potential for autocrine and paracrine effects on stellate cells. Hepatology, 27 (1998), pp. 472-480
Yanagisawa, M. et al. A novel potent vasoconstrictor peptide produced by vascular endothelial cells. Nature 332, 411-415 (1988).
Zhao, L. et al. OutCyte: a novel tool for predicting unconventional protein secretion. Sci Rep 9, 19448 (2019).

1-13. (canceled)

14. A method of treating or preventing muscle fibrosis comprising administering an endothelin receptor antagonist or a pharmaceutical composition comprising an endothelin receptor antagonist to a patient having a muscle fibrosis.

15. The method according to claim 14, wherein said antagonist is an antagonist of endothelin type-B receptor or an antagonist of endothelin type-A receptor.

16. The method according to claim 14, wherein said antagonist is a dual endothelin receptor antagonist, an antagonist which binds selectively to endothelin type-B receptor, or an antagonist which binds selectively to endothelin type-A receptor.

17. The method according to claim 14, wherein said endothelin receptor antagonist is selected from Bosentan, TAK044, SB209670, A192621, BQ788, IRL2500, atrasentan, K-8794, RES7011, Ro 46-8443, macitentan, aprocitentan, ambrisentan, BQ123, sitaxsentan, an antibody, an antibody mimetic or an aptamer directed to human endothelin type-A receptor or an antibody, antibody mimetic or an aptamer directed to human endothelin type-B receptor.

18. The method according to claim 17, wherein said endothelin receptor antagonist is selected from BQ123, Bosentan or BQ788.

19. The method according to claim 14, wherein the patient has a myopathy, a muscle fibrosis not associated with a myopathy, or is an elderly patient without muscular dystrophy but suffering from achalasia.

20. The method according to claim 19, wherein:
a) the myopathy is a muscular dystrophy, oculopharyngeal muscular dystrophy, Duchenne muscular dystrophy, inclusion body myositis, facioscapulohumeral muscular dystrophy, Becker muscular dystrophy, Limb-girdle muscular dystrophy, Distal muscular dystrophy, congenital muscular dystrophy, Emery-Dreifuss muscular dystrophy; or
b) the muscle fibrosis not in connection with myopathy is a muscle fibrosis developed in patient without muscular dystrophy but suffering from dysphagia.

21. The method according to claim 19, wherein the patient is an elderly person without muscular dystrophy but suffering from achalasia.

22. The method according to claim 14, wherein the patient has a pharyngeal muscle fibrosis.

23. The method according to claim 22, wherein the patient has an oculopharyngeal muscular dystrophy or inclusion myositis, or is an elderly person suffering from achalasia.

24. The method according to claim 14, wherein the endothelin receptor antagonist or pharmaceutical composition is administered in an amount from 50-500 mg/day.

25. The method according to claim 24, wherein the endothelin receptor antagonist or pharmaceutical composition is administered in an amount from 50-400 mg/day.

26. The method according to claim 24, wherein the endothelin receptor antagonist or pharmaceutical composition is administered in an amount from 50-300 mg/day.

27. The method according to claim 24, wherein the endothelin receptor antagonist or pharmaceutical composition is administered in an amount from 60-250 mg/day.

28. The method according to claim 24, wherein the endothelin receptor antagonist or pharmaceutical composition is administered in an amount from 80-250 mg/day.

29. A kit-of-parts comprising or consisting of:
at least one endothelin receptor antagonist, and
a means for carrying out a gene therapy or cellular therapy or pharmacological therapy for use in the treatment of a myopathy or a muscular dystrophy,
for simultaneous, separate or sequential use in the treatment or the prevention of muscle fibrosis in patients suffering from a myopathy or a muscular dystrophy.

30. The kit according to claim 29, wherein said endothelin receptor antagonist is an antagonist of endothelin type-B receptor, an antagonist of endothelin type-A receptor, a dual endothelin receptor antagonist, an antagonist which binds selectively to endothelin type-B receptor, or an antagonist which binds selectively to endothelin type-A receptor.

31. An in vitro method for diagnosing muscle fibrosis in a human subject suspected of suffering from muscle fibrosis, said method comprising:
determining the circulating endothelin level in a biological sample obtained from said human subject; and
comparing said circulating endothelin level with a reference level to correlate said circulating endothelin level to the presence or absence of muscle fibrosis in said human subject.

32. A method for evaluating the effectiveness of an anti-fibrotic treatment, said method comprising:
determining the circulating endothelin level in a biological sample obtained from a human subject under a treatment of muscle fibrosis; and
comparing said circulating endothelin level with an initial level to evaluate the effectiveness of said treatment of muscle fibrosis.
